(19) United States
(12) Patent Application Publication
(10) Pub. No.: US 2011/0257452 A1
(43) Pub. Date: Oct. 20, 2011

(54) Regenerable Composite Catalysts for Hydrocarbon Aromatization

(75) Inventors: Olga Khabashesku, Houston, TX (US); James R. Butler, League City, TX (US); Darak Wachowicz, Friendswood, TX (US); Callum Bailey, Seabrook, TX (US)

(73) Assignee: Fina Technology, Inc., Houston, TX (US)

(21) Appl. No.: 13/045,572

(22) Filed: Mar. 11, 2011

Related U.S. Application Data
(63) Continuation-in-part of application No. 12/763,279, filed on Apr. 20, 2010.

(51) Int. Cl. C07C 2/00; B01J 29/04; B01J 29/00; B82Y 30/00

(52) U.S. Cl. 585/418; 585/407; 502/100; 502/60; 502/343; 502/300; 502/355; 502/353; 977/902

(57) ABSTRACT

A composite catalyst for aromatization of hydrocarbons includes a molecular sieve catalyst and metal dehydrogenation catalyst present as discrete catalysts in a physical admixture. The molecular sieve catalyst can be a zeolite and the metal dehydrogenation catalyst can be in the form of a nanostructure, such as zinc oxide nanopowder. The catalyst can convert hydrocarbon feedstocks, such as alkanes and alkenes, to aromatics and can be regenerated in-situ.

### CROSS-REFERENCE TO RELATED APPLICATIONS

The present application is a continuation-in-part of U.S. patent application Ser. No. 12/763,279 filed on Apr. 20, 2010.

### FIELD

The present invention generally relates to catalysts used in aromatization of hydrocarbons such as olefins.

### BACKGROUND

The conversion of hydrocarbons to aromatics is of considerable importance because it provides a route for producing high value aromatic hydrocarbons, such as benzene, toluene and xylenes, from less expensive feedstocks, such as propane, methane, butane and olefins. For instance, the cost of benzene can undergo periodical changes, and higher-priced periods can place a hardship on the styrene and polystyrene businesses. Providing aromatics, especially benzene, from relatively inexpensive feedstocks is an economically attractive way to produce precursors for styrene monomer.

The process by which light alkanes and alkenes are converted into aromatic products is a catalytic aromatization reaction, which is a complex reaction that can include the steps of dehydrogenation, oligomerization, and aromatization.

Metal-containing catalysts, in which the metal is incorporated into a zeolite structure by some process, such as ion exchange or impregnation are known. However, wide swings in catalytic activity may occur in the case of the metal impregnated catalyst as metal is lost from the pore structure. Another drawback is the undesirably high probability of plugging pores with coke when metal is incorporated into a zeolite structure.

In addition to these problems, prior art catalysts have also generally contained expensive components, such as specialized zeolite and platinum, which in addition to their being expensive materials, have shown to produce low conversion rates and high levels of coking Furthermore, the aromatization process generally favors low weight hour space velocities, and relatively high volumes of catalyst must be used for process operation. Thus, due to the relatively high volumes of catalyst that are used a lower cost catalyst is desirable.

In light of the above it is desirable to have efficient catalysts in terms of the selectivity and stability for aromatization reactions that avoid the above-described problems.

### SUMMARY

In a non-limiting embodiment, either by itself or in combination with any other aspect of the invention, the present invention is directed towards a composite catalyst, containing a molecular sieve catalyst and a metal dehydrogenation catalyst, for the conversion of hydrocarbons, such as light alkanes and alkenes, to aromatics.

In a non-limiting embodiment, either by itself or in combination with any other aspect of the invention, the invention is a composite catalyst for the aromatization of paraffins and olefins, having a molecular sieve catalyst and a metal dehydrogenation catalyst that is present as a nanostructure. The two co-catalysts can be present as discrete catalysts in a physical admixture. The molecular sieve catalyst can be a zeolite. The metal dehydrogenation catalyst can be zinc oxide and can be present in the form of a nanopowder. The composite catalyst can be promoted with another metal, such as rhenium, niobium, and/or gallium. The zinc oxide can be impregnated with a gallium oxide promoter. The composite catalyst can be used for the aromatization of methane, low alkanes, such as C₂-C₆ alkanes, and can be used with LPG as the feedstock. The composite catalyst can also be used for the aromatization of olefins, such as C₂-C₈ alkenes. The composite catalyst can be regenerated in-situ by hydrogen and water vapor stripping at the reaction temperature.

In a non-limiting embodiment, either by itself or in combination with any other aspect of the invention, the present invention is a process for the aromatization of hydrocarbons that includes introducing a hydrocarbon feedstock into a reaction chamber, passing the feedstock over a composite aromatization catalyst at reaction conditions effective to provide a product containing aromatic hydrocarbons, and regenerating the catalyst in-situ. The feedstock can be C₂-C₆ alkanes, alkenes and combinations thereof. The feedstock can further include steam and can further include methane. The composite catalyst can include a metal dehydrogenation catalyst such as zinc oxide nanopowder and a molecular sieve or zeolite and can additionally include promoters such as rhenium, niobium, and gallium. The reaction conditions can include a temperature of from 350° C. to 650° C., a pressure of from 30 to 300 psi, and weight hourly space velocity of from 0.3 hr⁻¹ to 10 hr⁻¹. The regeneration can include hydrogen and water vapor stripping at the reaction temperature. The process may further include collecting methane, ethane, and propane in a recycle stream and feeding the recycle stream to reaction chamber for aromatization.

In a non-limiting embodiment, either by itself or in combination with any other aspect of the invention, the present invention is a process for the aromatization of olefins that includes introducing an alkene feedstock into a reaction chamber, passing the feedstock over a composite aromatization catalyst at reaction conditions effective to provide a product containing aromatic hydrocarbons, and regenerating the catalyst in-situ. The feedstock can be C₃-C₈ alkenes. The feedstock may include methane in combination with olefins. The feedstock may include alkanes in combination with olefins. The feedstock may include a mixture of butanes and butenes. The feedstock may further include steam. The composite catalyst can include a metal dehydrogenation catalyst such as zinc oxide nanopowder and a molecular sieve or zeolite and can additionally include promoters such as rhenium, niobium, and gallium. The reaction conditions can include a temperature of from 350° C. to 650° C., a pressure of from 30 to 300 psi, and weight hourly space velocity of from 0.3 hr⁻¹ to 10⁻¹. The regeneration can include hydrogen and water vapor stripping at the reaction temperature. The process may further include collecting methane, ethane, and propane in a recycle stream and feeding the recycle stream to reaction chamber for aromatization.

Other possible embodiments include two or more of the above aspects of the invention. In an embodiment the method includes all of the above aspects and the various procedures can be carried out in any order.

### BRIEF DESCRIPTION OF DRAWINGS

FIG. 1 is a graph of test results from an experiment using a composite catalyst containing only zinc and zeolite.

FIG. 2 is a graph of test results from an experiment using a composite catalyst containing only zinc and zeolite.

FIG. 3 is a graph of test results from an experiment using a composite catalyst containing zinc, zeolite and rhenium.

FIG. 4 is a graph of test results from an experiment using a composite catalyst containing zinc, zeolite, rhenium and gallium.

FIG. 5 is a graph of test results from an experiment using a composite catalyst containing zinc, zeolite and rhenium.

FIG. 6 is a graph of test results from an experiment using a regenerated composite catalyst containing zinc, zeolite, rhenium and gallium.

FIG. 7 is a graph of test results from an experiment using composite catalyst containing niobium, rhenium, and a mix of ZSM-5 and Y-type zeolite.

FIG. 8 is a graph of test results from an experiment using a butane feed and a composite catalyst containing zinc, zeolite, rhenium and niobium.

FIG. 9 is a graph of test results from an experiment using a butane feed and a composite catalyst containing zinc, zeolite, rhenium and niobium.

FIG. 10 is a graph of test results from an experiment using a butane and a isobutane feed and a composite catalyst containing zinc, zeolite, rhenium, niobium, and lanthanum.

FIG. 11 is a graph of conversions and selectivities observed at different space velocities at 520° C. with isobutene feed.

### DETAILED DESCRIPTION

The present invention is a composite catalyst for the aromatization of paraffins, olefins, or a combination thereof. In an alternate embodiment, the invention is a process for the aromatization of paraffins, olefins, or a combination thereof. The composite catalyst can include a molecular sieve catalyst and a metal dehydrogenation catalyst that is present as a nanostructure. The two co-catalysts can be present as discrete catalysts in a physical admixture.

The process by which light alkanes and alkenes are converted into aromatic products is an aromatization reaction, which can be a complex, multistage reaction. Schematically, alkane aromatization can be considered as a three-stage process, including the steps of dehydrogenation, oligomerization, and aromatization. All three processes can occur simultaneously, but can be better understood if described as occurring sequentially. Aromatization of olefins has generally been performed over bifunctional zeolite catalysts that contain active sites of two different types, active sites for dehydrogenation and active sites for oligomerization.

The first step converts the alkane feedstock into olefins and occurs via one of two reactions. Either a hydrogen-carbon bond on an alkane is broken, to form a hydrogen atom and the corresponding olefin, or carbon-carbon bond fissure takes place to form a lighter alkane and an olefin. Over a zeolite at reaction temperatures for aromatization, the latter reaction can be favored, due to carbon-carbon bonds possessing lower bond energy than carbon-hydrogen bonds. This reaction can undesirably produce alkanes in the aromatization product, which decreases the total selectivity to aromatic products.

The second stage of aromatization is alkene interconversion, including alkene isomerization, oligomerization and cracking steps, to form cyclic napthenes. In the third stage, the cyclic napthenes are dehydrogenated to their corresponding aromatic hydrocarbons in a sequence of cyclization and hydrogen transfer steps. As a consequence of the bimolecular hydrogen transfer mechanisms, formation of aromatics (hydrogen-deficient hydrocarbons) is balanced by formation of alkanes, which decrease the maximum possible selectivity to aromatics. If lower alkanes are produced in the initial stage, by carbon-carbon bond fissure, the maximum possible selectivity to aromatics is even further reduced.

When a metal oxide dehydrogenation catalyst is employed, however, the first stage of aromatization favors the breaking of hydrogen-carbon bonds, in which less lower alkane side-products are produced. Thus, the aromatics selectivity can be increased significantly by using bifunctional, metal-containing zeolite catalysts. The reviews of studies of the catalysts with various metals show that Zn and Ga-containing zeolites can be considered efficient catalysts in terms of the selectivity and stability for aromatization reactions.

Prior art describes the use of metal-containing zeolite catalysts, in which the metal is incorporated into the zeolite structure by some process, such as ion exchange or impregnation. However, swings in catalytic activity may occur in the case of the metal impregnated catalyst as metal is lost from the pore structure. Another drawback is the high probability of plugging pores with coke when the metal is incorporated into the zeolite structure.

In addition to these problems, prior art catalysts have also generally contained expensive components, such as specialized zeolite and platinum, which in addition to their being expensive materials, have shown to produce low conversion rates and high levels of coking. Furthermore, the aromatization process generally favors low space velocities and relatively high volumes of catalyst must be used for process operation. Thus, a lower cost catalyst is desirable.

Reaction 1 shows a reaction sequence for the aromatization of paraffins. Aromatization can include the steps of dehydrogenation, oligomerization, and aromatization or cyclization (another dehydrogenation reaction). The composite catalyst of the present invention contains a dehydrogenation catalyst and a molecular sieve catalyst. The dehydrogenation catalyst is responsible for the two dehydrogenation steps shown in the reaction sequence of Reaction 1, while the molecular sieve catalyst is responsible for the oligomerization step. With the introduction of olefins in a feedstream the aromatization of olefins, via oligomerization and aromatization as in the last two steps in the reaction sequence, can occur along with the aromatization of paraffins.

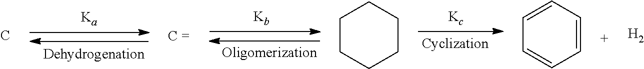

The dehydrogenation catalyst can be a metal oxide, such as zinc oxide. The molecular sieve catalyst can be a zeolite. One desirable zeolite is ZSM-5, and it can be used in the protonated form. The zinc oxide is desirably not incorporated into the structure of the zeolite. Rather, the two components of the composite catalyst are present as discrete catalysts in a physical admixture. The zinc oxide can be a zinc oxide nanopowder, to increase the interface with the zeolite. The process of combining the discrete catalysts can be any known in the art, whereby the catalysts remain distinct from one another. In the examples given below, the composite catalyst is formed by mixing zeolite with zinc oxide nanopowder by tumbling, followed by pressing, crushing and sieving.

An advantage to using a physical admixture of discrete catalysts is that the composite catalyst can be simpler in formulation and lower in cost than an impregnated catalyst system. Aromatization processes generally favor low weight hour space velocities, and therefore relatively high volumes of the catalyst must be used for the process operation. Thus, a lower cost catalyst is desirable. Another advantage is that use of the composite catalyst may not be as affected by wide swings in catalytic activity which may occur in the case of the metal impregnated catalyst as metal is lost from the pore structure. Additionally, the composite catalyst of the present invention has a lower probability of plugging pores with coke than when metal is incorporated into a zeolite structure.

The composite catalyst can further contain various promoters, such as other metals, for instance, gallium, rhenium, niobium, lanthanum, or some combination thereof. Some of these promoters, such as rhenium, can reduce the rate of catalyst coking by controlling the production of naphthalenes. The promoting metal can be added in any suitable manner known in the art, such as non-limiting examples of supported on a substrate or an inert support, added to a binder, placed on or within the zeolite or the dehydrogenation catalyst, such as by ion exchange, incipient wetness impregnation, pore volume impregnation, soaking, percolation, wash coat, precipitation, and gel formation. The promoters can range from 0.1 wt % to 10.0 wt % or more of the final catalyst, optionally from 0.1 wt % to 8.0 wt %, optionally from 0.1 to 5.0 wt %.

A catalyst component or the composite catalyst having a substrate that supports a promoting metal or a combination of metals can be prepared in any suitable manner, many of which are known in the art.

The various elements that make up the catalyst components or the composite catalyst can be derived from any suitable source, such as in their elemental form, or in compounds or coordination complexes of an organic or inorganic nature, such as carbonates, oxides, hydroxides, nitrates, acetates, chlorides, phosphates, sulfides and sulfonates. The elements and/or compounds can be prepared by any suitable method, known in the art, for the preparation of such materials.

The term “substrate” as used herein is not meant to indicate that this component is necessarily inactive, while the other metals and/or promoters are the active species. On the contrary, the substrate can be an active part of the catalyst. The term “substrate” would merely imply that the substrate makes up a significant quantity, generally 10% or more by weight, of the entire catalyst. The promoters individually can range from 0.01% to 60% by weight of the catalyst, optionally from 0.01% to 50%. If more than one promoter is combined, they together generally can range from 0.01% up to 70% by weight of the catalyst. The elements of the catalyst components or the composite catalyst can be provided from any suitable source, such as in its elemental form, as a salt, as a coordination compound, etc.

In one embodiment, the catalyst can be prepared by combining a substrate with at least one promoter element. Embodiments of a substrate can be a molecular sieve, from either natural or synthetic sources. Zeolites can be an effective substrate, can be commercially available, and are well known in the art. One desirable zeolite is ZSM-5, and it can be used in the protonated form. Another form of zeolite that may be used is Y-type zeolite. Y-type zeolite promotes dehydrogenation reactions, such as the dehydrogenation of alkanes to olefins that is the first step of the aromatization reaction. In one embodiment, the substrate can be a combination of ZSM-5 zeolite and Y-type zeolite, combined, for instance, in a 50:50 ratio. Alternate molecular sieves also contemplated are zeolite-like materials such as for example crystalline silicoaluminophosphates (SAPO) and the aluminophosphates (ALPO). The catalyst can undergo a methylcellulose treatment along with optional other extrusion aids and/or binder materials to increase porosity of the final catalyst extrudate and enhance activity.

The present invention is not limited by the method of catalyst preparation, and all suitable methods should be considered to fall within the scope herein. Particularly effective techniques are those utilized for the preparation of solid catalysts wherein a molecular sieve is used as a substrate and one or more promoter elements are added. Conventional methods include co-precipitation from an aqueous, an organic, or a combination solution-dispersion, impregnation, dry mixing, wet mixing or the like, alone or in various combinations. In general, any method can be used which provides compositions of matter containing the prescribed components in effective amounts. According to an embodiment the substrate is charged with promoter via an incipient wetness impregnation. Other impregnation techniques such as by soaking, pore volume impregnation, or percolation can optionally be used. Alternate methods such as ion exchange, wash coat, precipitation, and gel formation can also be used. Various methods and procedures for catalyst preparation are listed in the technical report Manual of Methods and Procedures for Catalyst Characterization by J. Haber, J. H. Block and B. Dolmon, published in the International Union of Pure and Applied Chemistry, Volume 67, Nos 8/9, pp. 1257-1306, 1995, incorporated herein in its entirety.

When slurries, precipitates or the like are prepared, they will generally be dried, usually at a temperature sufficient to volatilize the water or other carrier, such as from 100° C. to 250° C., with or without vacuum. Irrespective of how the components are combined and irrespective of the source of the components, the dried composition can be calcined in the presence of a free oxygen-containing gas, usually at temperatures between about 300° C. and about 900° C. for from 1 to 24 hours. The calcination can be in an oxygen-containing atmosphere, or alternately in a reducing or inert atmosphere.

The addition of a support material to improve the catalyst component and/or the composite catalyst physical properties is possible within the present invention. Binder material, extrusion aids or other additives can be added to the catalyst composition or the final catalyst composition can be added to a structured material that provides a support structure. For example, the catalyst component and/or the composite catalyst can include an alumina or aluminate framework as a support. Upon calcination these elements can be altered, such as through oxidation which would increase the relative content of oxygen within the final catalyst structure. The combination of the catalyst component and/or the composite catalyst of the present invention combined with additional elements such as a binder, extrusion aid, structured material, or other additives, and their respective calcination products, are included within the scope of the invention.

The prepared composite catalyst can be ground, pressed, sieved, shaped and/or otherwise processed into a form suitable for loading into a reactor. The reactor can be any type known in the art, such as a fixed bed, fluidized bed, or swing bed reactor. Optionally an inert material, such as quartz chips, can be used to support the composite catalyst bed and to locate the composite catalyst within the bed. Depending on the composite catalyst, a pretreatment of the composite catalyst may, or may not, be necessary. For the pretreatment, the reactor can be heated to elevated temperatures, such as 200° C. to 900° C. with an air flow, such as 100 mL/min, and held at these conditions for a length of time, such as 1 to 3 hours. Then, the reactor can be brought to the operating temperature of the reactor, for example 150° C. to 600° C., or optionally down to atmospheric or other desired temperature. The reactor can be kept under an inert purge, such as under a nitrogen or helium purge.

Sulfiding consists of the process of depositing sulfur on a catalyst. Sulfiding is known in the art and all suitable sulfiding methods should be considered to fall within the scope herein for the catalyst components and/or the composite catalyst. A generalized sulfiding procedure involves a sulfur-bearing agent and hydrogen in contact with the catalyst at an elevated temperature. The hydrogen reacts with the sulfur-bearing agent to produce hydrogen sulfide (H₂S), which serves as the sulfiding medium. The H₂S reacts with the metallic catalyst, which gives up an oxygen to form water. The sulfur replaces the oxygen on the catalyst. The process generally follows a schedule of four stages that include: a) placing the catalyst and a sulfur-bearing agent, such as dimethyl sulfide or dimethyl sulfoxide, in a reactor that is purged of air and dehydrated, with or without vacuum, temperature can be in the range of 120° C. to 150° C.; b) hydrogen is introduced with the catalyst and sulfur-bearing agent and the temperature is increased, for example from 40° C. to 230° C.; c) sulfiding occurs in an atmosphere of H₂S, temperature can be in the range of 230° C. to 260° C.; d) sulfiding continues in an atmosphere of H₂S at an elevated temperature, such as in the range of 270° C. to 290° C. A minimum of four hours is typically necessary to complete the sulfiding process. In one example the steps of b), c) and d) each take approximately two hours to complete.

The catalyst components of the present invention, the dehydrogenation catalyst and a molecular sieve catalyst, are present as discrete catalysts in a physical admixture and can be present in a weight ratio of dehydrogenation catalyst and a molecular sieve catalyst, for example zinc oxide to zeolite, of from about 0.1 to 1, optionally from 0.2 to 0.8, optionally from 0.3 to 0.5.

In an embodiment the composite catalyst of the present invention can undergo in-situ regeneration, which can lower operating costs by decreasing the amount of time the reactor must be offline. The regeneration can be done by hydrogen and water vapor stripping at the reaction temperature. In an embodiment the composite catalyst of the present invention can undergo ex-situ regeneration.

In another embodiment, the invention is a process for the aromatization of paraffins to aromatic hydrocarbons. The process includes the steps of introducing an alkane feedstock into a reaction chamber, passing the feedstock over a composite aromatization catalyst at reaction conditions effective to provide a product containing aromatic hydrocarbons, and regenerating the catalyst in-situ, when necessary.

The alkane feedstock can be paraffins containing less than 10 carbon atoms. The feedstock can consist primarily of C₂-C₆ alkanes, but may also consist of methane in combination with C₂-C₆ alkanes. The alkane feedstock can be obtained from the side product of various hydrocarbons processing plants, for instance, the offgas of an FCC cracker. One source of alkane feedstock is liquid petroleum gas (LPG), which consists mainly of the propane and butane fraction and can be recovered from gas and oil fields and petroleum refining operations. Co-feed can contain hydrogen, water vapor, and methane. Since the catalyst can withstand steam at the temperatures used for this process, steam can be used as a co-feed to increase conversion while reducing coke formation. Carbon dioxide can also be used as a co-feed as a mild oxidant to remove coke from the catalyst surface. In one embodiment, the alkane feedstock consists primarily of butane, with an optional methane co-feed. Hydrocracking of intermediates formed during the first steps of the aromatization process is possible and can lead to formation of lighter molecules and loss of yield. Production of lower alkanes can be achieved by adjusting equilibrium with reaction conditions. Methane as a co-feed suppresses methane production by shifting the methane-producing cracking reaction equilibrium to the left.

In another embodiment, the invention is a process for the aromatization of olefins to aromatic hydrocarbons. The feedstock can consist primarily of C₃-C₈ alkenes, but may also include methane in combination with C₃-C₈ alkenes and may also include C₃-C₆ alkanes. For instance, the feedstock can contain butane, isobutane, butene, isobutylene, or some combination thereof, with an optional methane co-feed. Raffinate-1 can also be used in the aromatization feed. Butene feeds may be obtained via the dehydrogenation of bio iso-butanol. Co-feed can contain hydrogen, water vapor, and methane. Since the catalyst can withstand steam at the temperatures used for this process, steam can be used as a co-feed to increase conversion while reducing coke formation. The process includes the steps of introducing an alkene feedstock into a reaction chamber, passing the feedstock over a composite aromatization catalyst at reaction conditions effective to provide a product containing aromatic hydrocarbons, and regenerating the catalyst in-situ, when necessary.

The composite catalyst for the process can be a composite catalyst containing zinc oxide nanopowder and a zeolite, as described above.

The reaction chamber can house any suitable catalyst system, such as a fixed catalyst bed, a moving bed or a fluidized bed. Single or multiple catalyst beds can be used, and the reactor can be a swing reactor.

The reaction can take place at a temperature of from 350° C. to 650° C., optionally from 400° C. to 600° C., optionally from 450° C. to 550° C. The pressure can be in the range of from 3 psig to 300 psig, optionally from 3 psig to 150 psig, optionally from 3 psig to 50 psig. The weight hourly space velocity can be from 0.3 to 50 hr⁻¹, optionally from 0.3 to 30 hr⁻¹, and optionally from 0.3 to 10 hr⁻¹.

The reaction products can be processed and separated by cooling or other standard recovery or separation techniques. The products of aromatization can include large amounts of hydrogen, which can be used for refining or petrochemical processing.

Hydrocarbons present in the off-gas can be recycled and used as co-feed in the aromatization reaction. Hydrocarbons that may be recycled include ethane, propane, other lower alkanes, as well as unconverted butanes and butenes, and combinations thereof.

Regeneration of the composite catalyst can be performed in-situ by hydrogen and water vapor stripping at the reaction temperature to prolong the catalyst life on stream.

The following examples are given to provide a better understanding of the present invention and are not intended to limit the scope of the invention in any way.

### EXAMPLES 1-6

Composite catalysts containing zeolite and zinc oxide nanopowder were prepared and tested in a reactor for the aromatization of alkanes. Two types of H-ZMS-5 zeolite, CBV-150 with silica to alumina ratio 150, and CBV-80 with silica to alumina ratio 80, were obtained from Zeolyst Company and used to make the test catalysts. The zinc oxide nanopowder that was used was less than 100 nm particle size, purchased from Aldrich.

The first two composite catalysts contained only zinc and zeolite, with one catalyst containing CBV-150 and the other containing CBV-80. These two catalysts, CBV-150/ZnO and CBV-80/ZnO, were prepared by mixing zeolite with zinc oxide nanopowder by tumbling followed by pressing, crushing and sieving to 40/60 mesh. The zinc oxide nanopowder was approximately 10 wt % of the mixed composition.

The other three composite catalysts contained some amount of rhenium or a combination of rhenium and gallium. Ammonim perrhenate and gallium nitrate hydrate, purchased from Aldrich, were used as the precursors for these promoting metals. For the rhenium and gallium containing catalyst, ammonium perrhenate was dissolved in deionized water and mixed with 10 g of silica nanopowder (15 ηm particle size) to obtain 2% Re (metal) loading on silica. The mixture was dried at 120° C. and calcined at 550° C. for 3 h. 2.5 g of ZnO nanopowder was mixed to insipient wetness with a solution of 0.973 g of gallium nitrate hydrate in 2.5 ml of water. The mixture was dried at 120° C. and calcined at 550° C. for 3 h. ZnO impregnated with Ga was mixed with 16.27 g of CBV-80 and 5 g of silica containing 2 wt % of Re. The final catalyst contained 3wt % Ga loading and 0.5 wt % of Re loading.

After the composite catalyst was prepared, the mixture was pressed, crushed and sieved to 40/60 mesh. For each test, 14 g of the composite catalyst was loaded into a stainless steel tubular reactor. The temperature was brought up to 460° C., and the reactor was left for 12 hours at this temperature under a hydrogen flow of 100 cc/min, to pre-reduce rhenium oxide to Re⁰. The hydrogen flow was then switched to a nitrogen flow of 50 cc/min and butane feed was introduced at 0.5 ml of liquid n-butane/min flow rate. Liquids exiting the reactor were condensed by passing the flow through water chiller at 8° C. and separated from off-gas in the glass receptacle bottle installed on scale. The off-gas flow rate was measured by wet test meter and analyzed by micro GC. The liquid effluent flow rate was monitored by scale readings. Liquids were analyzed by GC, which was calibrated to quantify naphthalenes content.

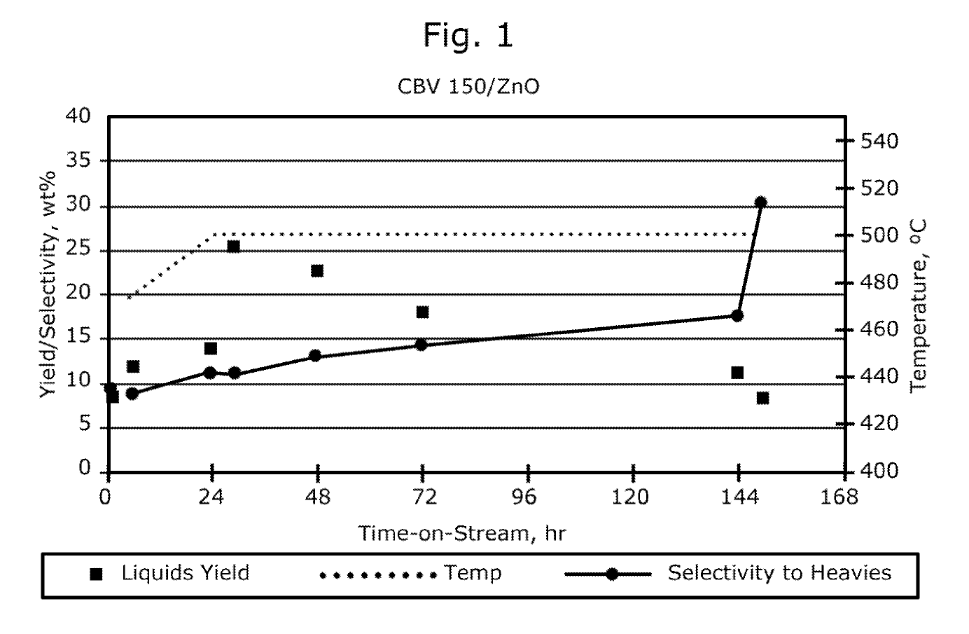

FIGS. 1-6 show the yield (wt %) of liquid aromatic products and the selectivity to heavies in conversion of n-butane, as they vary with temperature and time on stream (TOS).

### EXAMPLE 1

FIG. 1 shows the results of the test run done with the composite catalyst containing CBV-150 and zinc oxide nanopowder. The yield to liquid aromatics ranged from a little less than 10 wt % of the starting butane, to a little more than 25 wt %, with the peak yield occurring at 24 hrs time on stream (TOS). The selectivity to heavies in the liquid effluent increased from about 10 wt % to about 30 wt %. Aromatics yield results show that a composite catalyst containing nanopowder ZnO and ZSM-5 zeolite with Si/Al ratio 150 is capable of producing aromatic yields up to 25 wt % of the reaction product stream composition, not considering unreacted feed. The same results show that coking of the catalyst is occurring from the loss of activity and increasing concentration of heavy aromatics in the effluent. The results of this test are shown in Table 1 below.

TABLE 1

| TOS | T | Liquids Yield | Heavies C9+ |
|---|---|---|---|
| hours | C. | wt % | wt % |
| 1 | 450 | 8.9 | 9.4 |
| 6 | 475 | 11.7 | 8.8 |
| 24 | 500 | 13.9 | 1.1 |
| 29 | 500 | 25.2 | 11.1 |
| 48 | 500 | 22.9 | 12.8 |
| 72 | 500 | 18 | 14.3 |
| 144 | 500 | 11.3 | 17.6 |
| 149 | 500 | 8.2 | 30.25 |

### EXAMPLE 2

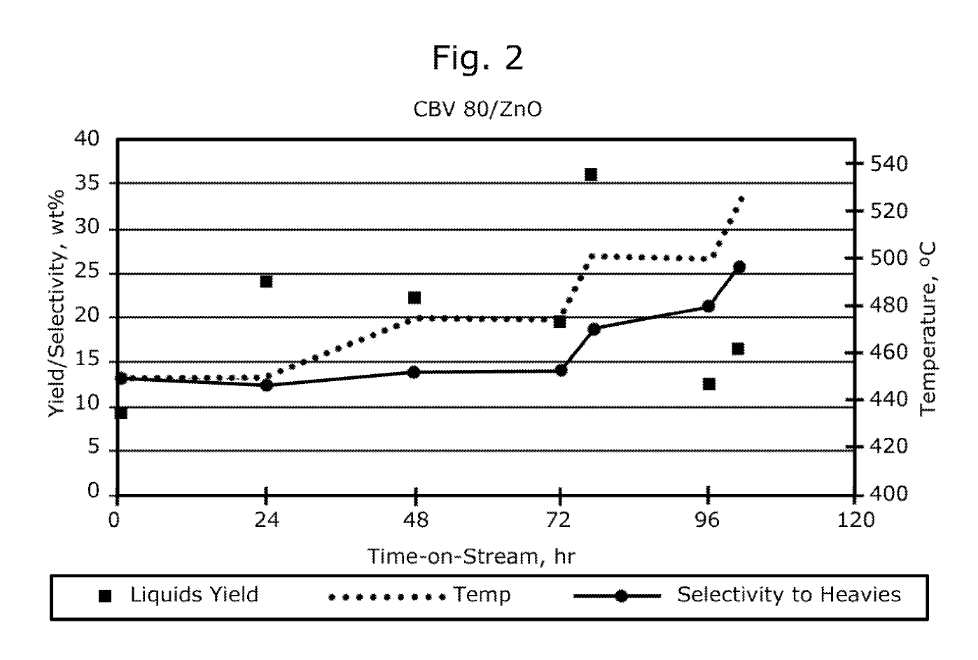

FIG. 2 shows the results of the test run done with the composite catalyst containing CBV-80 and zinc oxide nanopowder. The selectivity to liquid aromatics ranged from a little less than 10 wt % to 36 wt % of the reaction product composition, not considering unreacted butane, with the peak in selectivity occurring at 76 hrs TOS. The selectivity to heavies increased from about 10 wt % to about 30 wt %. Results from this test show that with the increase of the ZSM-5 zeolite acidity by using CBV-80 zeolite with Si/Al ratio of 80, the yield of liquid aromatics products can be increased up to 35 wt % of the reaction product stream composition. However, the rate of coke deposition appears high, which causes rapid catalyst deactivation. The results of this test are shown in Table 2 below.

TABLE 2

| TOS | T | Liquids Yield | Heavies C9+ |
|---|---|---|---|
| hours | C. | wt % | wt % |
| 0.5 | 450 | 9.8 | 9.4 |
| 24 | 450 | 24.8 | 8.8 |
| 48 | 470 | 23.1 | 12.8 |
| 72 | 470 | 20.1 | 14.3 |
| 76 | 500 | 36 | 12.8 |
| 96 | 500 | 13.1 | 17.6 |
| 100 | 520 | 17 | 30.25 |

### EXAMPLE 3

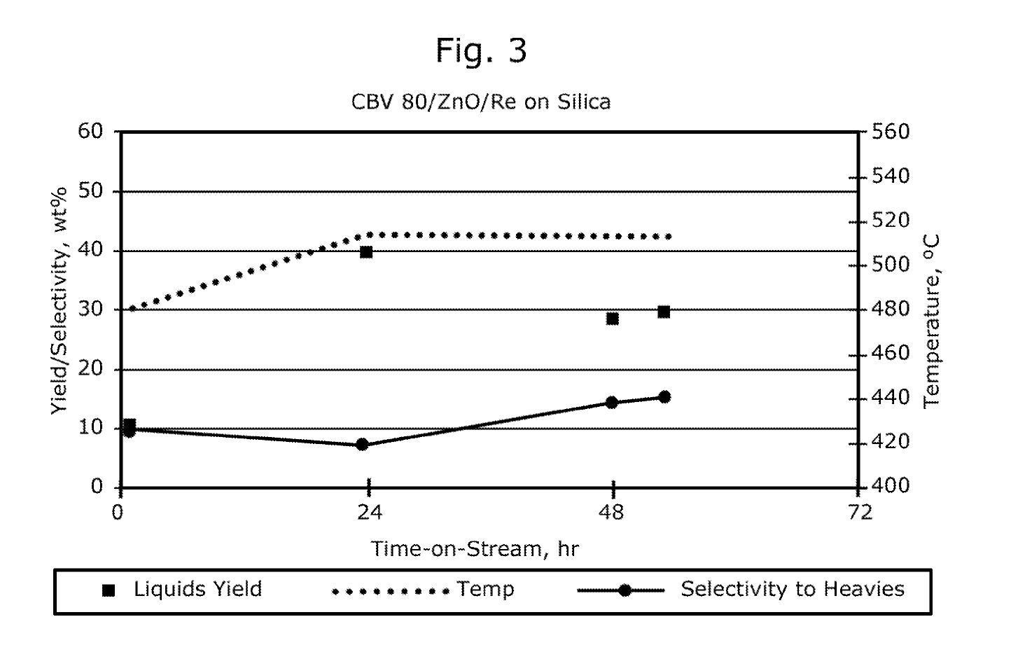

FIG. 3 shows the results of the test run done with the composite catalyst containing CVB-80 and zinc oxide nanopowder with 0.5 wt % Re loading. The selectivity to liquid aromatics ranged from about 10 wt % to 40 wt % of the reaction product stream composition, not considering unreacted butane, with the peak in selectivity occurring at 24 hrs TOS. The selectivity to heavies increased from about 10 wt % to about 15 wt %. Table 3 results illustrate the benefit of adding Re deposited on silica nano-powder to the composite catalyst containing ZSM-5 CBV-80 zeolite and nanopowder of ZnO. Aromatic products yield has reached 30-40 wt % of the reaction product stream composition, not considering unreacted butane. In this composite catalyst composition, Re was deposited on nano-silica. This experiment shows that addition of Re was beneficial for keeping the concentration of heavy aromatic products of the reaction product stream composition, not considering unreacted butane, below 15 wt %, while achieving greater than 25 wt % selectivity to liquid aromatics, which is an improvement in comparison with results shown in Tables 1 and 2. The results of this test are shown in Table 3 below.

TABLE 3

| TOS | T | Liquids Yield | Heavies C9+ |
|---|---|---|---|
| hours | C. | wt % | wt % |
| 0.5 | 480 | 10.1 | 9.8 |
| 24 | 513 | 40 | 8.1 |
| 48 | 513 | 30 | 14.2 |
| 52 | 513 | 31 | 15.1 |

### EXAMPLE 4

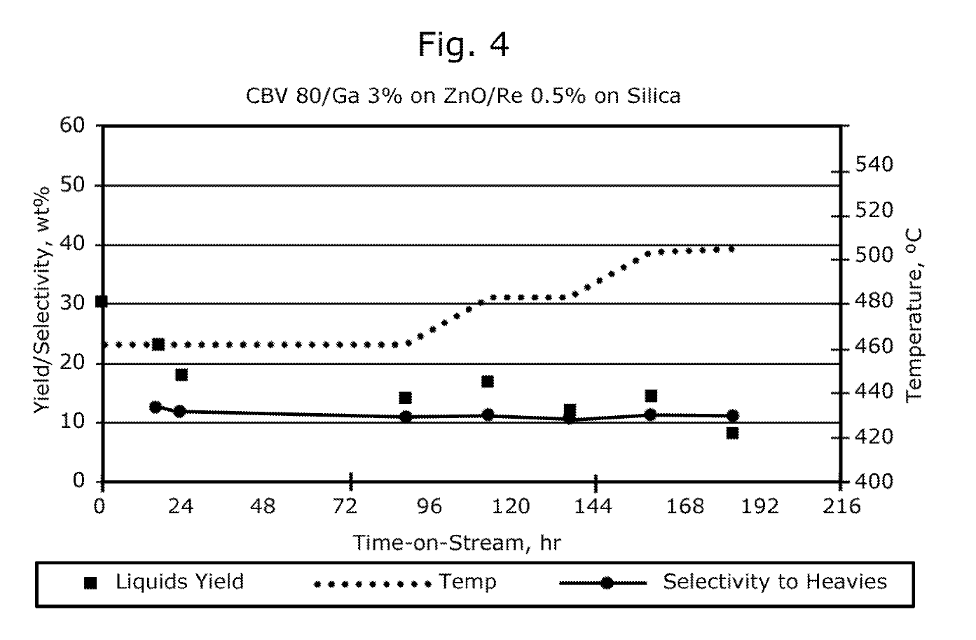

FIG. 4 shows the results of the test run done with the composite catalyst containing CVB-80 zinc oxide nanopowder composite catalyst with 3.0 wt % Ga loading and with 0.5 wt % Re loading. The yield of liquid products ranged from about 8 wt % to 32 wt % with the peak yield occurring at 0.5 hrs TOS. The selectivity to the liquid effluent heavies stayed relatively steady, decreasing from about 13 wt % to 11 wt % before rising to 12.3 wt % of the reaction product stream composition, not considering unreacted butane. These results demonstrate that the aromatic product yield for the Ga-containing catalyst did not show an increase comparing to the previous composition without Ga. Ga₂O₃ was present as a regular-sized powder, not a nano-structured material. This experiment also shows that addition of Re was beneficial for keeping concentration of heavy aromatic products below 15 wt % of the reaction product composition, which is an improvement in comparison with results shown in Tables 1 and 2. The results of this test are shown in Table 4 below.

TABLE 4

| TOS | T | Liquids Yield | Heavies C9+ |
|---|---|---|---|
| hours | C. | wt % | wt % |
| 0.5 | 450 | 32 | 13.1 |
| 24 | 450 | 24.1 | 12.5 |
| 80 | 450 | 14.8 | 11 |
| 118 | 480 | 12.5 | 12 |
| 138 | 480 | 13 | 12 |
| 158 | 500 | 14.9 | 12.3 |
| 178 | 500 | 8 | 12.3 |

### EXAMPLE 5

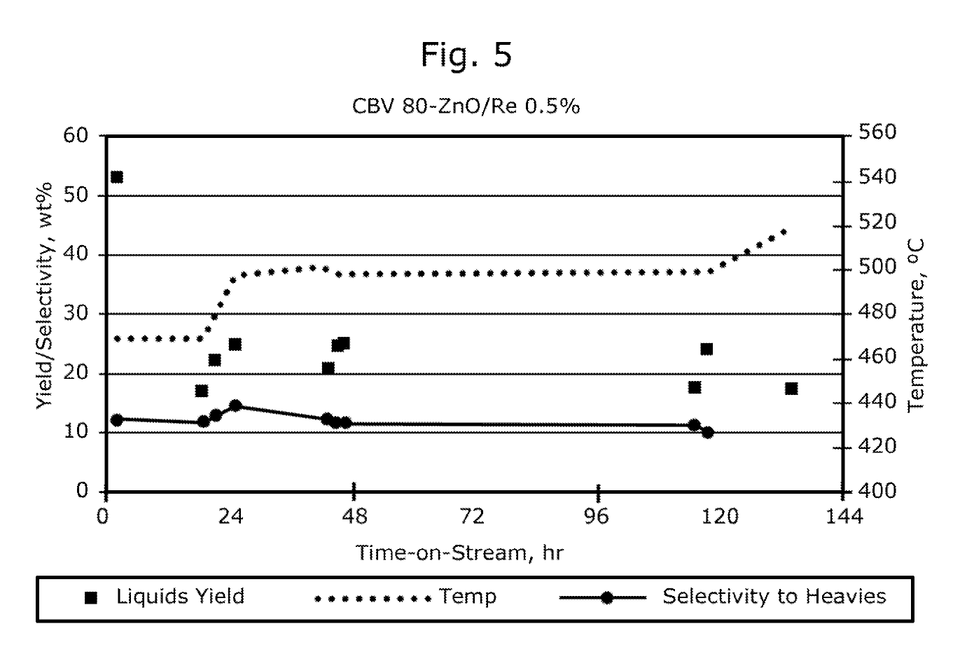

FIG. 5 shows the results of a test run done with a composite catalyst containing CBV-80 and zinc oxide nanopowder with 0.5 wt % Re loading deposited on the mixture of zeolite and zinc oxide nanopowder. The liquids yield ranged from 18 wt % to 52 wt % with the peak yield occurring at 0.5 hrs TOS. The selectivity to heavies in the liquid effluent stayed relatively steady, ranging from 10 wt % to 13 wt % of the reaction product stream composition, not considering unreacted butane. This example shows that in case of Re deposited on the mixture of ZSM-5 zeolite CBV-80 and nano-ZnO, the catalyst shows stable performance with heavies concentration in the liquid effluent staying at approximately 10 wt % of the reaction product stream composition, not considering unreacted butane, prolonging the catalyst's life on stream. The results of this test are shown in Table 5 below.

TABLE 5

| TOS | T | Liquids Yield | Heavies C9+ |
|---|---|---|---|
| hours | C. | wt % | wt % |
| 0.5 | 470 | 52 | 12 |
| 18 | 480 | 22 | 11.1 |
| 22 | 480 | 25 | 13.1 |
| 46 | 495 | 21 | 12 |
| 47 | 495 | 24.6 | 11.1 |
| 48 | 495 | 25.3 | 11.2 |
| 116 | 495 | 18 | 10.3 |
| 118 | 459 | 22 | 9.8 |

### EXAMPLE 6

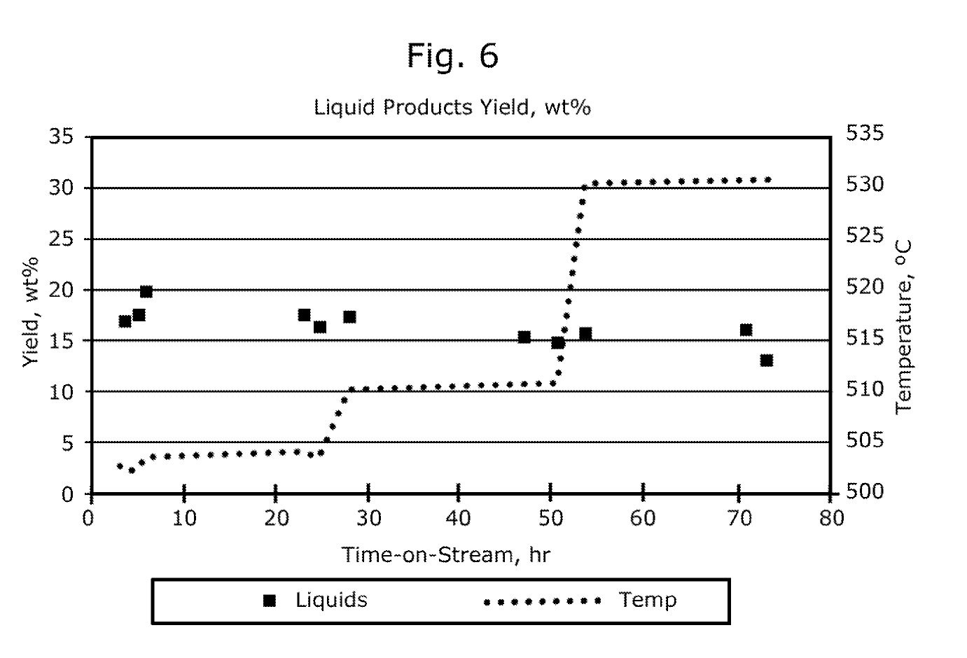

The composite catalyst used in Example 4, which is CVB-80 with 3.0 wt % Ga loading and zinc oxide nanopowder with 0.5 wt % Re loading was regenerated by calcination at 550° C. for 12 hours in an oven. The composite catalyst after regeneration was white colored and indications that substantially all coke deposits had been burned out. The regenerated composite catalyst was reloaded into the reactor and tested for butane aromatization at the same conditions as the fresh catalyst (Table 4, FIG. 4). The results of this test are shown in Table 6 below. As shown in Table 6 and FIG. 6, the composite catalyst regained activity and produced liquid aromatic products with yields reaching 15 wt % to 20 wt % of the reaction product stream composition, not considering unreacted butane. This indicates that the composite catalyst containing nanopowder ZnO can be regenerated by coke burnout, which can be done either by ex-situ or in-situ processes.

TABLE 6

| TOS, hr | T, C. | Conversion, wt % |
|---|---|---|
| 1 | 503.4 | 82.40 |
| 6 | 504.0 | 78.10 |
| 19 | 504.0 | 41.62 |
| 24 | 504.0 | 70.10 |
| 48 | 511.0 | 62.20 |
| 52 | 531.0 | 75.00 |
| 72 | 530.0 | 62.00 |

### EXAMPLE 7

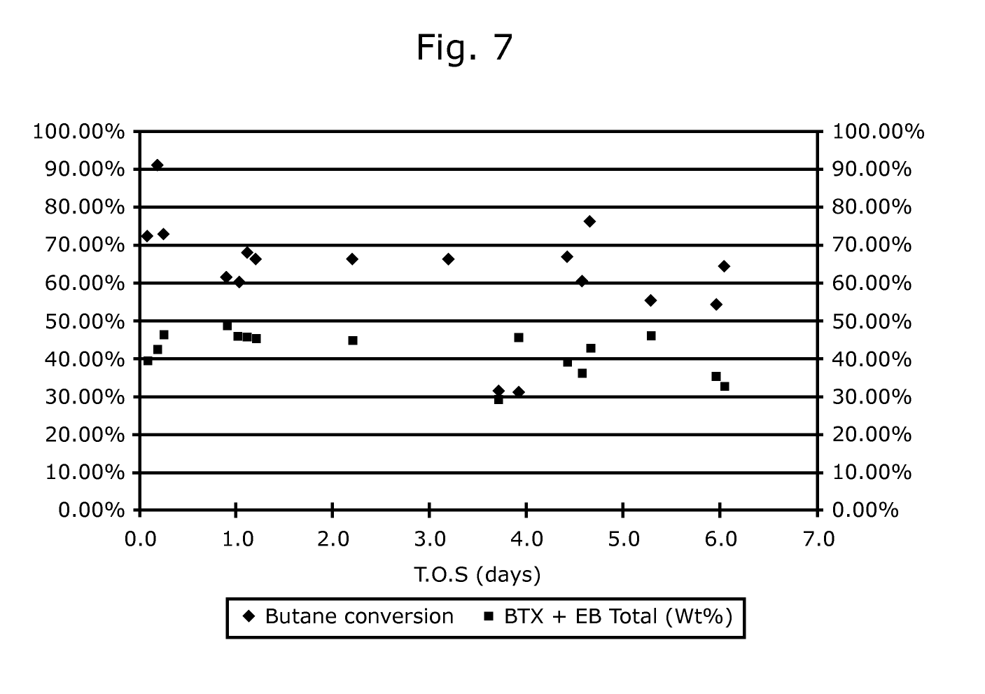

An aromatization catalyst was prepared and tested for the conversion of butane. The catalyst contained a mixed zeolite substrate, of ZSM-5 and Y-type zeolite in a 50/50 ratio. The catalyst also contained 5 wt % Nb and 0.5 wt % Re. FIG. 7 shows butane conversion and selectivity to BTEX (benzene, toluene, ethylbenzene, and xylene) over time-on-stream (TOS). The reaction over the mixed zeolite catalyst achieved selectivity to liquid products of 58 wt %, with BTEX selectivity 49%. Butane conversion ranged from ˜52 to 91 wt %. Catalyst sustained performance for five days.

Use of Y-type zeolite can enhance dehydrogenation function of the catalyst. A Y zeolite, CBV 780 with Si/Al ratio 80, was tested for dehydrogenation of butane. CBV780 has a very high surface area, 720 m²/g. ZSM-5 zeolite CBV 80 has surface area 400 m²/g. High surface area can be desirable in heterogeneous catalysis to increase catalyst-reactant interface and increase catalyst efficiency. Butane converted over Y-type zeolite produced liquid aromatics and gas containing a mixture of butenes, propene and ethylene with very low content of methane and ethane. Table 7 shows the selectivity to major products over the CBV780-based catalyst at low butane conversion. Selectivity to olefins produced by this catalyst is high with low selectivity to methane and ethane. A mixed zeolite catalyst containing Y-type zeolite can achieve high selectivity to BTEX due to the presence of the olefins in the feed formed over the Y zeolite.

TABLE 7
Selectivity to major products over CBV780-
based catalyst at low butane conversion.

| Average Temp. (° C.) | 511.2 |
|---|---|
| Butane Conversion, mol % | 8.44% |
| Gas Product Selectivity | wt % normalized |
| Hydrogen | 5.9% |
| Methane | 3.9% |
| Ethane | 0.0% |
| Other gas-phase alkanes | 1.3% |
| Gas-phase olefins | 88.8% |
| Total wt % | 100.0% |

### EXAMPLE 8

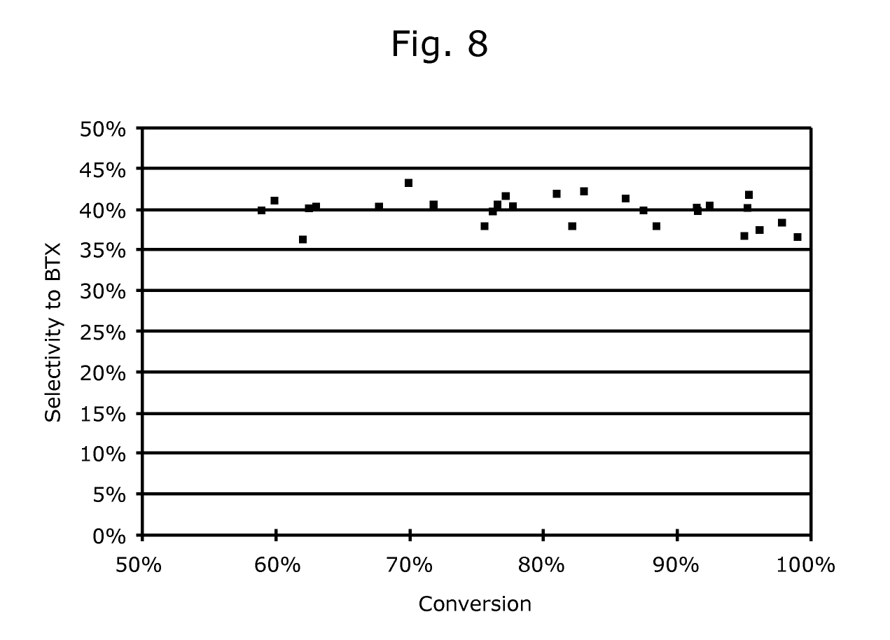

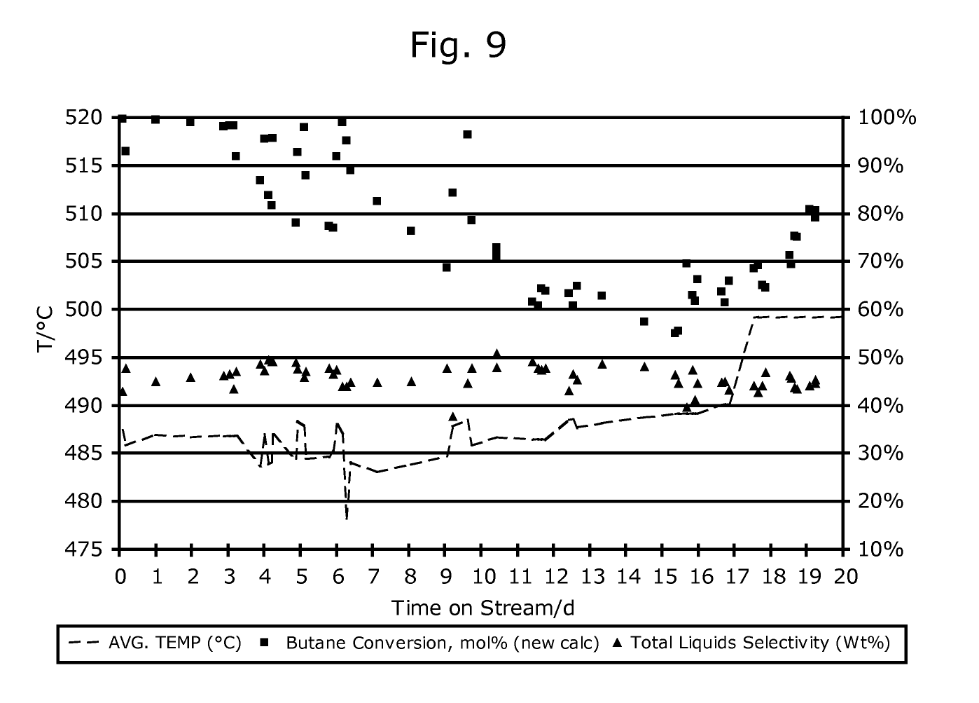

A composite catalyst was prepared containing 5 wt % niobium and 0.5 wt % rhenuim over CBV-80/nano-ZnO. The catalyst was first tested with a butane feed, the results of which are shown in FIGS. 8 and 9. The catalyst was on stream for 20 days and was regenerated two times with moist hydrogen stripping at the reaction temperature. The temperature of the catalyst bed was around 500° C. FIG. 8 shows selectivity to BTEX over butane conversion. FIG. 9 shows total liquids selectivity and butane conversion over time on stream. BTEX selectivity was from 40 wt % to 43 wt % over a wide range of butane conversions from 65 wt % to 95 wt %.

Methane was added as a co-feed with butane, in a methane to butane ratio varying from 0.80 to 3.31, and the co-aromatization was done over the same CBV-80/nano-ZnO/Nb5%/Re0.5% catalyst. The results are shown in Table 8. When methane is added as a co-feed, methane production is suppressed by shifting methane-producing cracking reaction equilibrium to the left. Methane used as a diluent for butane feed is activated, dehydrogenated and consumed in the aromatization process. Methane activation and conversion to aromatics occurred at methane to butane ratios above 2 and at temperatures above 500° C.

TABLE 8
Selectivities for methane co-aromatization with butane
versus Methane-to-Butane ratio and process temperature.

| | | | | | | | |
|---|---|---|---|---|---|---|---|
| Mass Balance | 100% | 99% | 101% | 100% | 107% | 97% | 93% |
| Avg. Temp (° C.) | 532 | 532 | 537 | 537 | 499 | 517 | 517 |
| CH₄/Butane ratio (mol) | 2.62 | 3.28 | 3.27 | 3.27 | 0.80 | 2.65 | 3.31 |

Product Selectivity (wt %)

| | | | | | | | |
|---|---|---|---|---|---|---|---|
| Hydrogen | 4.1 | 4.4 | 4.5 | 4.4 | 3.3 | 4.5 | 4.7 |
| Methane | −20.9 | −32.1 | −33.6 | −35.6 | −6.4 | −25.4 | −36.0 |
| Ethane | 26.1 | 24.2 | 24.4 | 23.0 | 17.6 | 21.6 | 20.1 |
| C₃-C₅ Alkanes | 12.4 | 11.5 | 10.6 | 10.0 | 17.9 | 14.4 | 13.5 |
| Gas-phase Olefins | 11.1 | 14.2 | 15.9 | 16.3 | 7.1 | 7.7 | 9.7 |
| Gas Non-aromatics | 28.7 | 17.8 | 17.3 | 13.7 | 36.2 | 18.1 | 7.3 |
| Liquid Non-aromatics | −0.1 | −0.1 | −0.2 | −0.2 | −0.4 | −0.2 | −0.2 |
| Ethylbenzene | 0.9 | 0.9 | .2 | 1.2 | 0.9 | 0.8 | 1.4 |
| Toluene | 17.3 | 16.6 | 15.9 | 15.1 | 16.4 | 21.5 | 17.9 |
| Benzene | 7.8 | 7.5 | 6.8 | 6.5 | 6.0 | 9.5 | 7.7 |
| Xylenes | 11.8 | 11.3 | 11.7 | 11.1 | 9.6 | 10.7 | 13.2 |
| Heavies | 46.7 | 35.1 | 34.2 | 29.8 | 53.1 | 40.2 | 26.4 |
| Total Liquids Selectivity | 42.7 | 41.1 | 40.6 | 38.6 | 38.5 | 47.5 | 45.8 |
| BTEX Selectivity | 37.7 | 36.3 | 35.6 | 33.9 | 32.9 | 42.5 | 40.2 |

Nb (IV) species were observed in Nb 3d XPS spectra of activated catalysts which are considered responsible for strong dehydrogenation/hydrogenation activity in methane-butane mixture co-aromatization. Nb (IV) is stabilized by the formation of mixed oxides with rhenium in-situ in reducing atmosphere.

### EXAMPLE 9

A composite catalyst was prepared containing 5 wt % niobium, 0.5 wt % rhenium, and 1 wt % lanthanum over CBV-30/nano-ZnO. The catalyst was first tested using a butane feed; after three days on stream, the feed was switched to isobutane. The isobutane feed contained 95% isobutane with 5% butane.

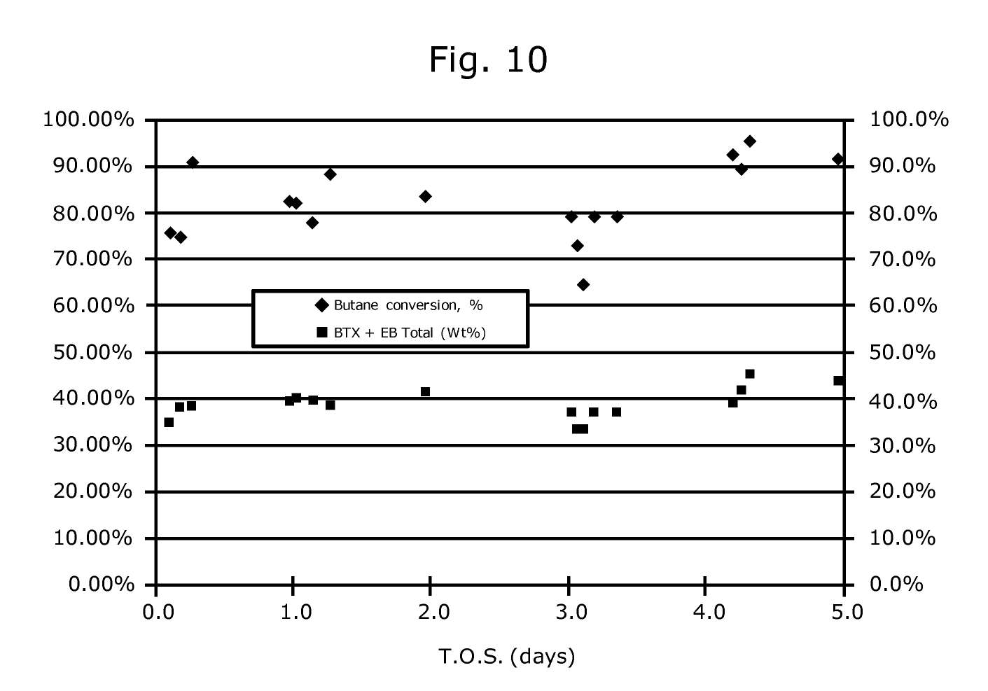

FIG. 10 shows conversion of butane and isobutane and selectivity to BTEX over the La-promoted composite catalyst. When the feed was switched to isobutane, the conversion of C4 increased from around 80 wt % to above 90 wt %, and BTEX selectivity improved from 33 wt % to 40 wt %. Table 9 shows effluent composition for butane and isobutane aromatization process. Effluent compositions with isobutane feed in comparison to butane showed higher percent of toluene and xylenes with lower benzene content.

TABLE 9
Effluent composition for butane and isobutane aromatization

Effluent composition, wt %

| Feed | Butane | Isobutane |
|---|---|---|
| Non-aromatics | 4.13 | 0.78 |
| Benzene | 20.82 | 15.11 |
| Toluene | 41.76 | 47.78 |
| Ethylbenzene | 2.29 | 0.70 |
| p-xylene | 6.49 | 6.81 |
| m-xylene | 12.25 | 14.77 |
| o-xylene | 5.20 | 6.71 |
| total xylenes | 23.94 | 28.29 |
| cumene | 0.12 | 0.06 |
| heavies | 4.22 | 4.50 |
| unknown | 0.83 | 2.25 |

### EXAMPLE 10

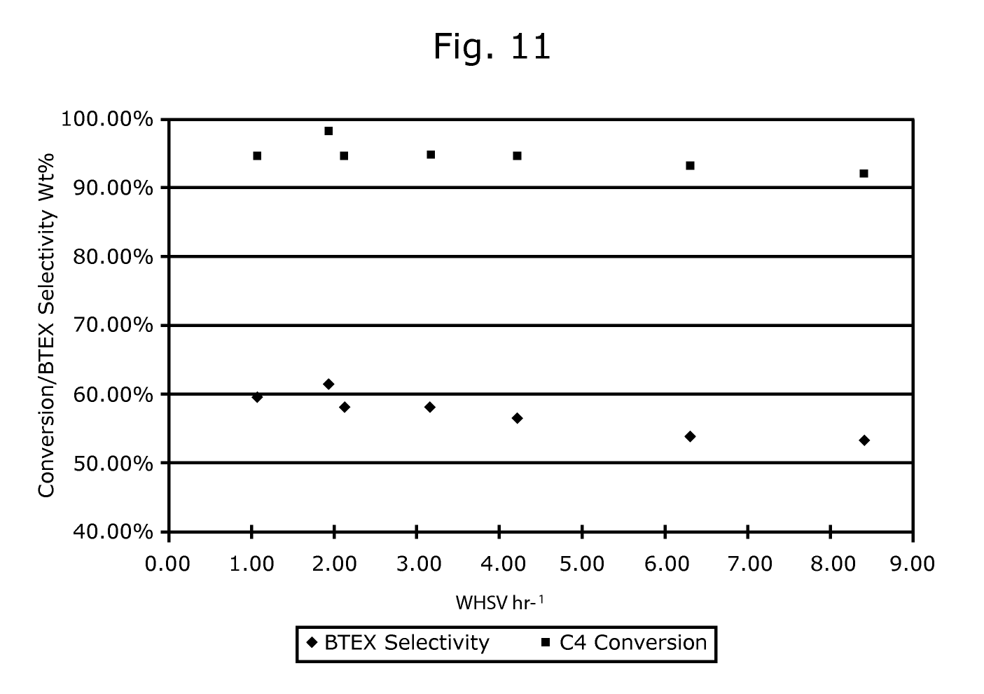

A composite catalyst, CBV-80/nanoZnO/Nb5%/Re0.5%, was used in the aromatization of an isobutene feed. BTEX selectivity was 61.4 wt % at 98% isobutene conversion for 100% isobutene feed with moisture (˜350 ppm concentration) co-feed over the CBV80 zeolite catalyst at 1.0 hr⁻¹ LHSV (0.5 ml/min) and 520° C. Liquid product selectivity was 69% with high BTEX content (90%). Low cracking was observed with selectivity to methane at about 4.4 wt % and ethane about 8.3 wt %. Increased selectivity to C9+ heavies was observed at 6.3 wt % with naphthalene selectivity 1.7 wt %. The space velocity was then increased. WHSV above 2.0 hr⁻¹ resulted in decreased selectivity to BTEX and increased production of non-aromatics, including olefins. FIG. 11 shows the conversions and selectivities observed at different space velocities at 520° C. with isobutene feed.

### EXAMPLE 11

A composite catalyst, CBV-80/nanoZnO/Nb5%/Re0.5%, was used in the aromatization of feeds containing butane, n-butene, isobutylene, and combinations thereof. One feed contained 1-butene and n-butane in a 60/40 ratio. One feed contained isobutylene and n-butane in a 60/40 ratio. One feed, which roughly simulated the composition of Raffinate-1, contained 1-butene, isobutylene, and n-butane in a 20/20/60 ratio. One feed contained 100% 1-butene. Table 10 shows the results of aromatization reactions of these four feeds when passed over the CBV-80/nanoZnO/Nb5%/Re0.5% catalyst.

TABLE 10
Conversion and selectivity of C4 feeds

| | | | 1-butene/ | |
|---|---|---|---|---|
| | 1-butene/ | i-butene/ | i-butene/ | 1- |
| | n-butane | n-butane | n-butane | butene |
| Feed Composition | 60/40 | 60/40 | 20/20/60 | 100 |
| Average Temp ° C. | 520 | 520 | 520 | 520 |
| Butane feed rate (ml/min) | 0.2 | 0.32 | 0.3 | — |
| 1-Butene feed rate (ml/min) | 0.3 | — | 0.1 | 0.5 |
| i-Butene feed rate (ml/min) | — | 0.48 | 0.1 | — |
| LHSV (hr⁻¹) | 1 | 1.6 | 1 | 1 |
| C4 Conversion, mol % | 86.9 | 80.4 | 72.8 | 85.27 |
| Butane Conversion, mol % | 66.1 | 50.6 | 54.5 | — |
| 1-Butene Conversion, | 100.0 | — | 100.0 | 100.0 |
| mol % | | | | |
| i-Butene Conversion, | — | 100.0 | 100.0 | — |
| mol % | | | | |
| Liquids Selectivity, wt % | 58.9 | 60.7 | 61.4 | 56.8 |
| BTEX Selectivity, wt % | 51.8 | 52.8 | 52.7 | 51.2 |

The various aspects of the present invention can be joined in combination with other aspects of the invention and the listed embodiments herein are not meant to limit the invention. All combinations of aspects of the invention are enabled, even if not given in a particular example herein.

Various terms are used herein, to the extent a term used is not defined herein, it should be given the broadest definition persons in the pertinent art have given that term as reflected in printed publications and issued patents.

The term “activity” refers to the weight of product produced per weight of the catalyst used in a process per hour of reaction at a standard set of conditions (e.g., grams product/gram catalyst/hr).

The term “conversion” refers to the weight percent of a reactant (e.g. butane) that undergoes a chemical reaction. X_{But}=cony of butane (wt %)=(Butᵢₙ−Butₒᵤₜ)/Butᵢₙ

The term “deactivated catalyst” refers to a catalyst that has lost enough catalyst activity to no longer be efficient in a specified process. Such efficiency is determined by individual process parameters. A deactivated catalyst generally requires process shut down in order for a regeneration procedure to be carried out.

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

As used herein the term “nanostructure” can refer to a material having at least one phase having at least one dimension of less than 100 nm.

The term “regeneration” refers to a process for renewing catalyst activity and/or making a catalyst reusable after its activity has reached a unacceptable/inefficient level. Examples of such regeneration may include passing steam over a catalyst bed or burning off carbon residue, for example.

The term “rhenium content of the catalyst” refers to the content of rhenium metal on the catalyst by weight as a percentage of the total catalyst weight or as a percentage of the weight of a specified portion of the catalyst. It is the weight of the Re elemental metal and not the entire weight of any possible Re containing compound, such as a Re oxide.

The term “selectivity” refers to the weight percentage that a particular product is out of the total of all the reaction products. The reaction products do not include unreacted feed. For example the selectivity to benzene would be the wt % of the reaction products that is benzene coming from the toluene that has reacted.

The term “zeolite” refers to a molecular sieve containing a silicate lattice, usually in association with some aluminum, boron, gallium, iron, and/or titanium, for example.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the aspects and embodiments disclosed herein are usable and combinable with every other embodiment and/or aspect disclosed herein, and consequently, this disclosure is enabling for any and all combinations of the embodiments and/or aspects disclosed herein. Other and further embodiments, versions and examples of the invention may be devised without departing from the basic scope thereof and the scope thereof is determined by the claims that follow.

What is claimed is:

1. A composite catalyst for the aromatization of hydrocarbons comprising:
a molecular sieve catalyst; and
a metal dehydrogenation catalyst;
wherein the molecular sieve catalyst and metal dehydrogenation catalyst are present as discrete catalysts in a physical admixture.

2. The catalyst of claim 1, wherein the molecular sieve catalyst is a zeolite.

3. The catalyst of claim 1, wherein at least a portion of the dehydrogenation catalyst is present as a nanostructure.

4. The catalyst of claim 1, wherein the metal dehydrogenation catalyst includes zinc oxide.

5. The catalyst of claim 4, wherein at least a portion of the zinc oxide is a zinc oxide nanopowder.

6. The catalyst of claim 1, wherein the composite catalyst is promoted with rhenium from 0.1 to 10.0 wt % of the composite catalyst.

7. The catalyst of claim 1, wherein the composite catalyst is promoted with gallium of at least 0.1 wt % of the composite catalyst.

8. The catalyst of claim 1, wherein the composite catalyst is promoted with niobium of at least 0.1 wt % of the composite catalyst.

9. The catalyst of claim 1, wherein the composite catalyst is promoted with rhenium from 0.1 to 10.0 wt % of the composite catalyst and also promoted with niobium from 0.1 to 10.0 wt % of the composite catalyst.

10. The catalyst of claim 1, wherein the composite catalyst is capable of converting olefins to aromatics.

11. The catalyst of claim 1, wherein the molecular sieve catalyst is a zeolite and the metal dehydrogenation catalyst includes a zinc oxide nanopowder that are present in ratios of zinc oxide to zeolite of from 0.1 to 1.

12. The catalyst of claim 1, wherein the composite catalyst can be regenerated in-situ by hydrogen and water vapor stripping at a reaction temperature suitable for the aromatization of hydrocarbons.

13. A composite catalyst for the aromatization of olefins comprising:
a molecular sieve catalyst; and
a metal dehydrogenation catalyst;
wherein the molecular sieve catalyst and metal dehydrogenation catalyst are present as discrete catalysts in a physical admixture;
wherein at least a portion of the dehydrogenation catalyst is present as a nanostructure;
wherein the composite catalyst is promoted with rhenium of at least 0.1 wt % of the composite catalyst.

14. The catalyst of claim 13, wherein at least a portion of the dehydrogenation catalyst is zinc oxide.

15. The catalyst of claim 13, wherein the composite catalyst is further promoted with gallium of at least 0.1 wt % of the composite catalyst.

16. The catalyst of claim 13, wherein the composite catalyst is further promoted with niobium of at least 0.1 wt % of the composite catalyst.

17. A process for the aromatization of hydrocarbons comprising:
introducing a hydrocarbon feedstock into a reaction chamber;
passing the feedstock over a composite aromatization catalyst at reaction conditions effective to provide a product containing aromatic hydrocarbons;
wherein the composite aromatization catalyst comprises a molecular sieve catalyst and metal dehydrogenation catalyst present as discrete catalysts in a physical admixture.

18. The process of claim 17, wherein at least a portion of the dehydrogenation catalyst is present as a nanostructure.

19. The process of claim 17, wherein the feedstock comprises C₂-C₈ alkenes.

20. The process of claim 17, wherein the feedstock additionally includes water vapor.

21. The process of claim 17, wherein the feedstock comprises methane.

22. The process of claim 17, wherein the feedstock comprises a mixture of alkanes and alkenes.

23. The process of claim 17, wherein the molecular sieve catalyst is a zeolite and the metal dehydrogenation catalyst is a zinc oxide nanopowder that are present in the composite aromatization catalyst in ratios of zinc oxide to zeolite of from 0.1 to 1.

24. The process of claim 17, wherein the composite aromatization catalyst is promoted with rhenium of at least 0.1 wt % of the composite catalyst.

25. The process of claim 17, wherein the composite aromatization catalyst is promoted with gallium of at least 0.1 wt % of the composite catalyst.

26. The process of claim 17, wherein the composite aromatization catalyst is promoted with niobium of at least 0.1 wt % of the composite catalyst.

27. The process of claim 17, wherein the reaction conditions include a temperature of from 350° C. to 650° C.

28. The process of claim 17, wherein the reaction conditions include a pressure of from 3 to 300 psi.

29. The process of claim 17, wherein the reaction conditions include a weight hourly space velocity of from 0.3 to 50 hr⁻¹.

30. The process of claim 17 further comprising;
collecting methane, ethane, and propane in a recycle stream, and introducing the recycle stream to the reaction chamber.

31. The process of claim 17 further comprising;
regenerating the composite aromatization catalyst in-situ.

32. The process of claim 31, wherein the regeneration includes hydrogen and water vapor stripping at the reaction temperature.
